SEQUENCE LISTING (1 sequence)

SEQ ID NO: 1 - protein, 20 aa, Artificial Sequence
  a peptide to which there is antigen specificity
GSKLKDPELSLKGTQHIMQA

(19) United States
(12) Patent Application Publication
(10) Pub. No.: US 2005/0186204 A1
(43) Pub. Date: Aug. 25, 2005

(54) Use of antibodies against FLT-1 for the treatment of osteoporosis

(76) Inventors: Peter Carmeliet, Blanden (BE); Desire Collen, Winksele (BE); Roger Bouillon, Winksele (BE); Gertrudis Carmeliet, Blanden (BE)

Correspondence Address: TRASK BRITT, P.O. BOX 2550, SALT LAKE CITY, UT, 84110, US

(21) Appl. No.: 11/023,278

(22) Filed: Dec. 27, 2004

Related U.S. Application Data
(63) Continuation of application No. PCT/EP03/50275, filed on Jun. 27, 2003.

(30) Foreign Application Priority Data
Jun. 28, 2002 (EP) 02077591.2

(51) Int. Cl. A61K 39/395

(52) U.S. Cl. 424/143.1

(57) ABSTRACT

This invention relates to antagonists of the placental growth factor receptor and signaling thereof, pharmaceutical compositions containing such antagonists and the use of such antagonists to prevent bone loss or bone mass and to enhance bone healing including the treatment of conditions which present with low bone mass and/or bone defects in vertebrates, and particularly mammals, including humans.

### CROSS-REFERENCE TO RELATED APPLICATION

This application is a continuation of PCT International Patent Application No. PCT/EP2003/050275, filed on Jun. 27, 2003, designating the United States of America, and published, in English, as PCT International Publication No. WO 2004/002525 A1 on Jan. 8, 2004, the contents of the entirety of which is incorporated by this reference.

### FIELD OF THE INVENTION

This invention relates generally to biotechnology, and, more particularly, to antagonists of the placental growth factor receptor and signaling thereof, pharmaceutical compositions containing such antagonists and the use of such antagonists to prevent bone loss or bone mass and to enhance bone healing including the treatment of conditions which present with low bone mass and/or bone defects in vertebrates, and particularly mammals, including humans.

### BACKGROUND OF THE INVENTION

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated. The elderly are at greatest risk of osteoporosis, and the problem is therefore predicted to increase significantly with the aging of the population. Worldwide fracture incidence is forecasted to increase three-fold over the next 60 years, and one study estimated that there will be 4.5 million hip fractures worldwide in 2050. Women are at greater risk of osteoporosis than men. Women experience a sharp acceleration of bone loss during the five years following menopause. Other factors that increase the risk include smoking, alcohol abuse, a sedentary lifestyle and low calcium intake. There are currently two main types of pharmaceutical therapy for the treatment of osteoporosis. The first is the use of anti-resorptive compounds to reduce the resorption of bone tissue. Estrogen is an example of an anti-resorptive agent. It is known that estrogen reduces fractures. In addition, Black, et al. in EP 0605193A1 report that estrogen, particularly when taken orally, lowers plasma levels of LDL and raises those of the beneficial high density lipoproteins (HDL's). However, estrogen failed to restore bone back to young adult levels in the established osteoporotic skeleton. Furthermore, long-term estrogen therapy, however, has been implicated in a variety of disorders, including an increase in the risk of uterine cancer, endometrial cancer and possibly breast cancer, causing many women to avoid this treatment. The significant undesirable effects associated with estrogen therapy support the need to develop alternative therapies for osteoporosis that have the desirable effect on serum LDL but do not cause undesirable effects. A second type of pharmaceutical therapy for the treatment of osteoporosis is the use of anabolic agents to promote bone formation and increase bone mass. Although there are a variety of osteoporosis therapies there is a continuing need and a continuing search in this field of art for alternative osteoporosis therapies. In addition, there is a need for bone fracture healing therapies. Also, there is a need for therapy which can promote bone re-growth into skeletal areas where defects exist such as defects caused or produced by, for example, tumors in bone. Further, there is a need for a safer therapy with fewer side effects. In the art several studies have focused on mechanisms of osteoclast activation. For example Niida et al (1999) have shown that vascular endothelial growth factor (VEGF) has a positive activity on osteoclast recruitment. One interesting homologue of VEGF is Placental growth factor (PlGF) but its role in bone has been poorly studied (Persico M. G. et al, 1999, Curr Top Microbiol Immunol 237, 31-40). U.S. Pat. No. 5,919,899 describes PlGF and its use in the treatment of inflammatory disorders, wounds and ulcers. Several inhibitors for PlGF signaling, such as antibodies and tetrameric peptides binding on PlGF or antibodies binding on the PlGF-receptor, are known in the art and are disclosed in WO 01/85796. Matsumoto Y. et al (2001) 47^{th} annual meeting, Orthopaedic Research Society, February 25-28, San Francisco, Calif., also describe that VEGF stimulates the chemotaxis of osteoclast precursor cells via the Flt-1-PI3K-FAK pathway. However, since the latter results are carried out in vitro there is no indication whatsoever that antibodies against Flt-1 could be used in vivo to prevent osteoporosis. The present invention relates to the surprising finding that antagonists of the PlGF receptor can be used for the manufacture of a medicament to suppress disorders of bone resorption such as osteoporosis.

### DETAILED DESCRIPTION OF THE INVENTION

The invention provides a medicament for the treatment of osteoporosis in higher mammals exhibiting decreased cortical bone mineral density and preventing osteoporosis due to cortical bone mineral density reduction in such mammals. Also provided are pharmaceutical compositions useful in treating osteoporosis. In our previous studies, the PlGF gene was inactivated in the mouse genome via homologous recombination in embryonic stem (ES) cells (Carmeliet P., 2000, J. Pathol. 190, 387-405, Carmeliet P., 1999, Curr. Interv. Cardiol. Reports 1, 322-335 and Carmeliet P. and Collen D., 1999, Curr. Top. Microbiol. Immunol. 237, 133-158). PlGF (PlGF^{-/-}) deficient mice are viable and fertile, and do not exhibit apparent bone defects. However, herein it is shown that upon careful examination of bone histomorphometry, bone remodeling and biochemical analysis of these PlGF KO mice that PlGF plays an unexpected role in the process of bone resorption. It is shown that PlGF deficiency results in decreased bone resorption, low bone turnover and increased trabecular bone mass. Thus, the present invention shows that PlGF receptor antagonists can be used for the manufacture of a medicament for treatment of bone disorders and more specifically for the treatment of conditions where there is an enhanced bone resorption such as, for example, osteoporosis. In the present invention, a PlGF receptor antagonist is defined as a molecule binding on the PlGF receptor (also called VEGF-Receptor 1 (VEGFR-1) or also called Flt-1 receptor) and the antagonist is capable of interfering with the binding of PlGF to its receptor (VEGF-Receptor 1 or Flt-1) and the antagonist is capable of interfering with the signal transduction of the receptor. In a preferred embodiment, the antagonist is an antibody capable of binding to VEGFR-1 and wherein the antibody is capable of inhibiting the signal transduction of VEGFR-1. To screen for the candidate/test antibodies for example cell lines that express VEGFR-1 may be used and the signal transduction is monitored as described in detail in PCT International Publication WO 01/85796 which is herein incorporated by reference. The monitoring can be measured using standard biochemical techniques. Other responses such as activation or suppression of catalytic activity, phosphorylation (e.g., the tyrosine phosphorylation of the intracellular domain of the receptor) or dephosphorylation of other proteins, activation or modulation of second messenger production, changes in cellular ion levels, association, dissociation or translocation of signaling molecules, or transcription or translation of specific genes may also be monitored. These assays may be performed using conventional techniques developed for these purposes in the course of screening. Inhibition of PlGF binding to its cellular receptor Flt-1 may, via signal transduction pathways, affect a variety of cellular processes. Cellular processes under the control of the VEGFR-1/PlGF signaling pathway may include, but are not limited to, normal cellular functions, proliferation, differentiation, maintenance of cell shape, and adhesion, in addition to abnormal or potentially deleterious processes such as unregulated cell proliferation, loss of contact inhibition, blocking of differentiation or cell death. The qualitative or quantitative observation and measurement of any of the described cellular processes by techniques known in the art may be advantageously used as a means of scoring for signal transduction in the course of screening.

Thus, in one embodiment, the invention provides the use of receptor antagonists of PlGF, particularly antibodies against VEGFR-1, for the manufacture of a medicament to treat bone resorption disorders. Antagonists such as antibodies of the PlGF receptor can suppress the bone resorption in the bone resorption disorders. In a specific embodiment the bone resorption disorder is osteoporosis. With “suppression” it is understood that suppression of bone resorption can occur for at least 20%, 30%, 40%, 50%, 60%, 70%, 80%, 90% or even 100%.

By “molecules” it is preferentially meant antibodies. The term ‘antibody’ or ‘antibodies’ relates to an antibody characterized as being specifically directed against the receptor of PlGF (VEGFR-1 which is also designated as Flt-1) or any functional derivative thereof, with the antibodies being preferably monoclonal antibodies; or an antigen-binding fragment thereof, of the F(ab′)₂, F(ab) or single chain Fv type, or any type of recombinant antibody derived thereof. Preferably these antibodies, including specific polyclonal antisera prepared against VEGFR-1 or any functional derivative thereof, have no cross-reactivity to others proteins. Monoclonal antibodies can for instance be produced by any hybridoma liable to be formed according to classical methods from splenic cells of an animal, particularly of a mouse or rat immunized against VEGFR-1 or any functional derivative thereof, and of cells of a myeloma cell line, and to be selected by the ability of the hybridoma to produce the monoclonal antibodies recognizing VEGFR-1 or any functional derivative thereof which have been initially used for the immunization of the animals. The monoclonal antibodies may be humanized versions of the mouse monoclonal antibodies made by means of recombinant DNA technology, departing from the mouse and/or human genomic DNA sequences coding for H and L chains or from cDNA clones coding for H and L chains. Alternatively, monoclonal antibodies may be human monoclonal antibodies. Such human monoclonal antibodies are prepared, for instance, by means of human peripheral blood lymphocytes (PBL) repopulation of severe combined immune deficiency (SCID) mice as described in PCT International Application PCT/EP 99/03605 or by using transgenic non-human animals capable of producing human antibodies as described in U.S. Pat. No. 5,545,806. Also fragments derived from these monoclonal antibodies such as Fab, F(ab)′₂ and ssFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies or fragments thereof, can be modified for various uses. The antibodies can also be labeled by an appropriate label of the enzymatic, fluorescent, or radioactive type. Several antibodies against Flt-1 are known in the art which can be used for the manufacture of a medicament to treat osteoporosis. Antibodies against Flt-1 which can also be used in the invention for the manufacture of a medicament for treatment of osteoporosis comprise the antibody described in Lu D. et al (2001) Cancer Res. 61 (19) 7002) and a anti-human Flt-1 monoclonal antibody (Kyowa Hakko Kogyo Co, Ltd—U.S. 2003/0088075). In another specific embodiment single chain antibodies specific to Flt-1 can be used in the scope of the present invention. An example of such a single chain antibody specific to Flt-1 is described in U.S. Pat. No. 5,874,542 (Imclone Systems Incorporated).

Another embodiment is the use of monoclonal antibody against VEGFR-1. A preferred method to produce Anti-VEGFR-1 is for instance by priming rats, for instance Lewis rats (Harlan Sprague-Dawley Inc., Indianapolis, Ind.) with a subcutaneous injection of a antigen comprising a murine VEGFR-1 fragment for instance extracellular domain of VEGFR-1 fused to the Fc-fragment (VEGFR-1-Fc). Emulsified in suitable adjuvant, for instance complete Freund's adjuvant (Sigma). Rats have to receive booster intraperitoneal injections, preferably 4 such booster injections at 2-3-wk intervals with 100 mg of VEGFR-1-Fc. Recombinant human sVEGFR-1-Fc and mouse sVEGFR-1-Fc can be purchased from R&D Systems (Minneapolis, Minn., USA). Rats showing the highest titter of blocking antibody, for instance in a VEGF/VEGFR-1-Fc blocking assays, should consequently be boosted intravenously with such VEGFR-1 antigen (e.g., Flt-FC), preferably with a dose of about 50 mg. About five days later the splenocytes can be harvested and fused to mouse myeloma cells, preferably the P3-X63-Ag8.653 cells. Generation of hybridomas and subcloning was performed according to the current standard protocols available to artisans skilled in the art. Hybridomas secreting anti-VEGFR-1 can for instance be selected for binding to soluble VEGFR-1-Fc and negative binding to Fc protein alone in ELISA. The anti-VEGFR-1 can then be selected for inhibition of VEGFR-1-Fc/ligand binding as described below. The binding kinetics of anti-VEGFR-1 can be measured using a Biacore biosensor (Pharmacia Biosensor). Anti-VEGFR-1 can then be produced by culture of hybridoma cells in a suitable medium for instance serum-free medium and the Anti-VEGFR-1 can be purified from conditioned media for instance by a multi-step chromatography process. Assessment for purity is generally done by SDS-PAGE and immuno-reactivity in ELISA with a soluble VEGFR-1 receptor. A negative control rat IgG can be used for comparison. Protein concentration of antibodies is usually determined using the BCA method. The efficiency of such anti-VEGFR-1 to block binding of VEGFR-1 ligands to their receptor can be measured by a VEGFR-1/PlGF blocking assays in plates coated with PlGF. After sequential incubation with VEGFR-1-alkaline phosphatase (AP), preincubated with various concentrations of anti-VEGFR-1, and colorigenic substrate, it is possible to measure binding by microtiter plate reading at 405 nm. VEGFR-1-alkaline phosphatase (AP) is obtainable by fusing the extracellular domain of VEGFR-1 to human secretory alkaline phosphatase. Binding of anti-VEGFR-1 to the VEGFR-1 receptor, can be assessed by a standard binding assay for instance by coating microtiter plates by VEGFR-1-alkaline phosphatase and sequential incubation with various concentrations of anti-VEGFR-1, goat anti-rat IgG-HRP and colorigenic substrate to quantified binding by reading on a microtiter plate reader at 450 nm.

Several anti-VEGFR-1 antibodies in the art are available to the public. They are for the VEGF Receptor 1 antibodies such as Mouse monoclonal to human VEGF Receptor (ab9541) [Flt-1/EIC] of Abcam Inc. or Novus Biologicals; Anti-Flt-1(VEGFR1) (cat# 06-679) with antigen specificity against peptide (GSKLKDPELSLKGTQHIMQA (SEQ ID NO:______), residues 26-45 of human Flt-1(VEGFR1) of Upstate Charlottesville, Va. 22903 USA; (GEA8021-2 and GEA8021-2) of Genex Biosciences; (cat#RDI-FLT1abrX and cat#RDI-FLT1abrx-1) of Research Diagnostics Inc, Flanders N.J. 07836 US; Mouse anti-humanFLT-1 monoclonal antibody (cat#MAB1664) and rabbit ANTI-FLT-1 affinity purified polyclonal antibody (cat#AB3128) of Chemicon International Temecula, Calif. 92590, USA and Human Flt-1/VEGFR1 epitope Specific Rabbit Antibody (Cat. #RB-9049-P0, -P1, or -P, Cat. #RB-9049-R7 and Cat. #RB-9049-PCS) of Lab Vision Corporation, Calif. 94539 USA.

Production and Purification of Monoclonal Antibodies: 2.6,10,14-Tetramethylpentadecane (e.g., Pristane of Sigma, 0.5 ml) are intraperitoneally injected into Balb/c female mice (6 to 8 weeks old from the birth). After 10 to 20 days, cells of clones are (1×106 to 107 cells) suspended in PBS and intraperitoneally inoculated into the mice. After 7 to 10 days, the mice are sacrificed and subjected to an abdominal operation, from which produced ascitic fluid can be collected. The ascitic fluid is centrifuged to remove insoluble matters, and a supernatant was recovered and stored at −20° C. until purification. Consequently, IgG can be purified from the ascitic fluid supernatant described above by using Hi-Trap Protein-A antibody purification kit (available from Pharmacia, Roosendaal, N L). Namely, the ascitic fluid (2 ml) can be added with Solution A (1.5 M glycine, 3 M NaCl, pH 8.9, 8 ml), and filtrated with a filter for filtration having a pore size of 45 μm (Millipore). After that, an obtained filtrate can be applied to a column (column volume: 1 ml) charged with Protein Sepharose HP (produced by Pharmacia) sufficiently equilibrated with Solution A, and the column is washed with Solution A in an amount of 10-fold column volume. Subsequently, an IgG fraction can be eluted with Solution B (0.1 M glycine, pH 2.8) in an amount of 10-fold column volume. The eluted IgG fraction can be dialyzed against PBS. The monoclonal antibodies can be determined for their IgG subclasses by using the purified antibodies obtained in the foregoing, by means of a commercially available subclass-determining kit (tradename: Mono Ab-ID EIA Kit A, produced by Zymed). This method is based on the ELISA method.

The Inhibitory Activities of Monoclonal Antibodies can be tested for complete inhibition of binding of rPlGF to its VEGFR1 receptor. This, for instance, can be measured in an immunofunctional ELISA in which 96-well plates are coated with 100 μl of 1 μg/ml of rmFIt-1/Fc chimera overnight at room temperature in PBS. After blocking for 1 hour with 1% BSA in PBS, 100 μl of a mixture of 70 μl of hybridoma medium pre-incubated with 70 μl of recombinant mPlGF-2 at 10 ng/ml for 2 hours at room temperature is then applied to the plate. A standard of rmPlGF-2 ranging from 20 ng/ml to 156 pg/ml can be included (diluted in PBS-Tween.BSA-EDTA). Plates can then be incubated 1 hour at 370° C. and 1 hour at room temperature, washed 5 times with PBS-Tween and 100 pi of biotinylated goat anti-murine PlGF-2 at 200 ng/ml can applied for 2 hours at room temperature. After washing 5 times with PBS-Tween, 100 μl of avidin-HRP conjugate (Vectastorin ABC kit) can be applied for 1 hour at room temperature. After washing 5 times with PBS-Tween, the plate can be developed with 90 μl of o-phenylene diamine in citrate phosphate buffer pH 5.0 for 30 minutes and measured at 490 nm.

The monoclonal antibodies produced in animals may be humanized, for instance by associating the binding complementarily determining region (“CDR”) from the non-human monoclonal antibody with human framework regions—in particular the constant C region of human gene—such as disclosed by Jones et al. in Nature (1986) 321:522 or Riechmann in Nature (1988) 332:323, or otherwise hybridized.

A preferred embodiment for preparing of F(ab′)2 or monovalent Fab fragments is for instance as follows: In order to prepare F(ab′)2 fragments, the monoclonal antibody can be dialyzed overnight against a 0.1 mol/L citrate buffer (pH 3.5). The antibody (200 parts) is then digested by incubation with pepsin (1 part) available from Sigma (St. Louis, Mo., US) for 1 hour at 37° C. Digestion is consequently stopped by adding 1 volume of a 1 M Tris HCl buffer (pH 9) to 10 volumes of antibody. Monovalent Fab fragments can prepared by papain digestion as follows: a 1 volume of a IM phosphate buffer (pH 7.3) is added to 10 volumes of the monoclonal antibody, then 1 volume papain (Sigma) is added to 25 volumes of the phosphate buffer containing monoclonal antibody, 10 mmol/I L-Cysteine HCl (Sigma) and 15 mmol/L ethylene diaminetetra-acetic acid (hereinafter referred to as EDTA). After incubation for 3 hours at 37° C., digestion is stopped by adding a final concentration of 30 mmol/I freshly prepared iodoacetamide solution (Sigma), keeping the mixture in the dark at room temperature for 30 minutes. Both F(ab′)2 and Fab fragments can further be purified from contaminating intact IgG and Fc fragments using protein-A-Sepharose. The purified fragments can finally be dialyzed against phosphate-buffered saline (hereinafter referred to as “PBS”). Purity of the fragments can be determined by sodium dodecyl sulfate polyacrylamide gel electrophoresis and the protein concentration can be measured using the bicinchonicic acid Protein Assay Reagent A (Pierce, Rockford, Ill.).

In a specific embodiment it should be clear that the therapeutic method of the present invention for the suppression of bone resorption can also be used in combination with any other therapy known in the art for the suppression of enhanced bone resorption.

The term “medicament to treat” relates to a composition comprising molecules (antagonists) as described above, preferentially antibodies against VEGFR-1, and a pharmaceutically acceptable carrier or excipient (both terms can be used interchangeably) to treat diseases as indicated above. Suitable carriers or excipients known to the skilled artisan are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. The “medicament” may be administered by any suitable method within the knowledge of the skilled artisan. The preferred route of administration is parenteral. In parental administration, the medicament of this invention will be formulated in a unit dosage injectable form such as a solution, suspension or emulsion, in association with the pharmaceutically acceptable excipients as defined above. However, the dosage and mode of administration will depend on the individual. Generally, the medicament is administered so that the protein, polypeptide, peptide of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute.

In a specific embodiment, antibodies or functional fragments thereof that bind on VEGF-R1 and neutralize its signal transduction can be used for the manufacture of a medicament for the treatment of the above-mentioned disorders. Non-limiting examples include where the antibodies are humanized (Rader et al., 2000, J. Biol. Chem. 275, 13668) and more preferentially human antibodies are used as a medicament.

The following examples more fully illustrate preferred features of the invention, but are not intended to limit the invention in any way. All of the starting materials and reagents disclosed below are known to those skilled in the art, and are available commercially or can be prepared using well-known techniques.

### EXAMPLES

1. Examination of the Bone Phenotype of PlGF Knockout Mice

PlGF deficient mice were described before in Carmeliet P et al. (2001) Nature Medicine 7:575-583.

1.1. Bone Histomorphometry

Bones were processed for bone histomorphometry as previously described (Daci et al, J Bone Miner Res. 2000, 15:1510-1516). Briefly, the bones were embedded undecalcified in methylmetacrylate and 4 μm thick longitudinal sections were cut with a rotary microtome (RM 2155, Leica, Heidelberg, DE) equipped with a tungsten carbide 50° knife. Sections were stained according to Von Kossa to assess mineralized bone. The measurements were performed in a standardized area comprising most of the proximal tibial metaphysis, using a Kontron Image Analyzing System (Kontron Electronic, KS 400 V 3.00, Eching bei Munich, DE). All parameters comply with the recommendations of the Histomorphometry Nomenclature Committee of the American Society for Bone and Mineral Research (Parfitt et al, J. Bone Miner Res 2:595-610, 1987). For immunohistochemistry, bones were fixed in 2% paraformaldehyde in PBS, decalcified in EDTA and embedded in paraffin. Bone sections were immunostained for CD31 as described.

Results:
An increase of 18% in trabecular bone volume was measured in the proximal tibial metaphysis of newborn PlGF deficient mice compared to WT mice. This increase became more pronounced (+42%; p<0.05) in 12 weeks-old PlGF deficient mice.
Bone histomorphometric studies using double calcein labeling documented a significant decrease in both mineral apposition rate (MAR by 47%) and bone formation rate (BFR by 61%) in 12 weeks-old knockout mice compared with WT mice
No vascularization defects were observed in 12 and 16 week-old PlGF-/- mice, despite the pronounced increase in trabecular bone mass.

1.2. Bone Mineral Density (BMD) and Indices of Bone Remodeling

Trabecular bone mineral density (BMD) was measured in excised tibiae by peripheral quantitative computer tomography (pQCT) (XCT-960M; Nordland Medical Systems Inc.) as described (Dacio et al., cfr supra). Four cross-sections (one cortical at mid-diaphysis and three trabecular at the proximal epiphysis) were scanned, and the data was analyzed using a threshold value of 200 mg/cm³ to select for bone and to exclude soft tissue. Cortical and trabecular bones were separated by “concentric peel” with the inner core defined as trabecular bone.

Results: analysis by pQCT showed that the trabecular bone mineral density was increased in PlGF-deficient mice at 12 weeks (+30%; p<0.05), whereas cortical bone parameters were only minimally affected. These observations confirmed the histomorphometric data.

1.3. Biochemical Analysis

Serum osteocalcin was measured by the in-house RIA described previously (Bouillon et al. 1992 Clin. Chem 38:2055-2060). Collagen cross-links were quantitated according to an assay previously described (Daci et al. cfr supra). Serum osteocalcin levels measured in PlGF-deficient mice of different ages were on average 30% lower compared to WT mice (p<0.05). Urinary excretion of collagen cross-links was reduced in 12 weeks-old knockout mice by 26% (p<0.05).

These data show that deficiency of PlGF in mice results in decreased bone resorption, low bone turnover and increased trabecular bone mass, showing an important role for PlGF in the process of bone resorption.

2. Mouse Models for Osteoporosis

2.1 Apolipoprotein-E Deficient Mouse

An epidemiological correlation is suggested between osteoporosis and cardiovascular disease independent of age. The basis for this correlation is unknown. Atherosclerosis-susceptible mice receiving a high-fat diet develop osteoporosis as reflected in a decrease in bone mineral content and bone mineral density (Parhami et al. J Bone Miner Res 2001, 16, 182-188). Apolipoprotein-E deficient (ApoE^{-/-}) mice were obtained from Dr. J. Breslow (The Rockefeller University, New York, USA). Mice had a mixed genetic background of 75% C57B1/6 and 25% 129SvJ. Animals were weaned at 4 weeks of age and maintained on normal chow diet for 1 week, after which time they were fed the high fat/high cholesterol diet. For studying the role of the PlGF receptor (VEGF-R1), Apoe^{-/-} mice were intraperitoneally injected three times per week with antibodies against the VEGR1: 500 μg MF-1 for 5 weeks starting at 5 weeks. (MF-1 is a monoclonal antibody against Flt1 developed at Imclone Systems Incorporated, also described in U.S. Patent Application U.S. 2003/0108545, the contents of which are incorporated by this reference). The results show that both male and female ApoE deficient mice, fed on the high fat/high cholesterol diet, showed a decrease in trabecular content by 37% (p<0.05) and 12% respectively and a decrease in trabecular density by 42% (p<0.05) and 15% respectively. The decrease in both these parameters was either completely prevented in female mice or partially prevented in male mice (p<0.05) receiving anti-VEGF-R1 antibodies. Thus osteoporosis which develops in atherosclerosis-susceptible mice on a high-fat/high cholesterol diet can be (partially) prevented by interfering with VEGF-R1 activity.

2.2 Unloading-Induced Bone Loss Mouse Model

Physical inactivity contributes to the development of osteoporosis. The hypothesis is that bone loss induced by inactivity results from decreased bone formation and decreased blood flow, and corresponding hypoxia (Dodd, 1999, Am. J. Physiol. 277: C598-C602). Physical inactivity can be mimicked in mice by a “hindlimb-unloading” model. Bone histomorphometry and bone mineral density were measured as described herein above. The Histomorphometry shows that hindlimb unloading reduces trabecular bone volume in WT mice significantly by 50%, while this decrease is only 20% in PlGF null mice (no significant difference could be found in reduction of trabecular bone volume when the PlGF null mice were compared with PlGF null mice with full activity). pQCT analysis shows comparable results for bone mineral density. Thus PlGF deficiency protects mice from bone loss induced by physical activity.

3. Osteoclast Formation and Function

3.1. Assays for Osteoclast Formation and Function

Osteoclast formation was studied using co-cultures of primary osteoblasts and bone marrow cells, treated with 1,25dihydroxyvitamin D₃. Briefly, the marrow cavity of the tibiae from 6- to 8-week-old mice was flushed with α-MEM, cells were collected by centrifugation and nucleated cells counted using Türk's solution. In co-culture experiments, primary osteoblasts were plated at 2×10⁴ cell/well in a 48-well culture plate and 24 h later bone marrow cells were added at 10⁵ nucleated cells/well. Primary osteoblasts derived from the knockout or WT mice were co-cultured with the corresponding bone marrow cells. Co-cultures were treated with 2×10⁻⁸ M 1.25 vitamin D₃ or vehicle on day 1, day 3 and stopped at day 6. At the end of the co-culture period, adherent cells were rinsed with PBS, fixed with 4% formaldehyde in PBS for 10 min, treated with ethanol-acetone 50:50 (v/v) for 1 min, air-dried and stained for TRAP. Cells were incubated at room temperature in 0.1 M sodium acetate, pH 5.0 containing naphthol As-MX phosphate and fast red violet LB salt, in the presence of 10 mM sodium tartrate. The number or size of cells staining positively and containing 3 or more nuclei was determined. Anti-VEGF-R1 antibodies (MF-1) were added at 250 μg/ml/48 hours.

In order to determine osteoclast resorbing activity, PlGF-deficient and WT osteoclasts formed in vitro were cultured for 48 h on dentine slices and the resorbed surface was corrected for osteoclast number. To explore the role of PlGF on osteoclast migration, PlGF-deficient and WT osteoclasts were cultured in the upper chamber of culture inserts with collagen-gel coated membranes and their migration to the lower chamber was assessed. Osteoclast survival was studied by counting total osteoclast numbers at different time-points during a 72 h period in cultures of mature osteoclasts formed in vitro.

Results:

The total number of osteoclasts formed in bone marrow-osteoblast co-cultures of PlGF deficient mice was decreased with 10% (p<0.05) compared to WT co-cultures. When counting only the largest osteoclasts, their number was 50% lower in PlGF deficient co-cultures compared to WT co-cultures. In addition, the percentage of osteoclasts with more than 5 nuclei was decreased significantly. That PlGF participates in osteoclast formation by acting directly on osteoclast progenitors was further demonstrated by studying osteoclast formation in cultures of nonadherent bone marrow cells derived from the knockout and WT mice and treated with M-CSF (macrophage-colony stimulating factor) and RANKL (receptor activator of NF-kappaB). The number of osteoclasts formed in cell cultures derived from the knockout mice was markedly lower (42±4, n=4 vs. 423±15, n=4, p<0.001) compared with WT cultures.

The size of osteoclasts formed in WT bone marrow-osteoblast co-cultures: (1) without anti-VEGF-R1 antibodies: 14260 μm² and (2) with anti-VEGF-R1 antibodies (MF-1): reduction by 70±2% of controls; N=3; P<0.05.

Osteoclasts derived from knockout or WT mice resorbed dentine similarly, without difference among the two genotypes. No difference was observed among PlGF deficient and WT osteoclasts (12.3±2.4%, n=3 vs. 13.9±1.7%, n=3, respectively) in their ability to migrate and invade the collagen matrix. No difference was observed in the survival of PlGF-1- and WT osteoclasts (71±7%, n=3 vs. 70±2%, n=3, respectively) at 48 h, nor at any other time-point studied (24 h and 72 h). Osteoclast formation and especially the maturation of osteoclast precursors to large multinucleated TRAP positive cells is affected by PlGF as deficiency or blocking of its signal transduction with the application of antibodies against VEGF-R1 resulted in decreased size (and number) of osteoclasts. In addition the in vitro data indicate that activity of mature osteoclasts is not affected by PlGF deficiency.

4. Bone Resorption Assay Ex Vivo

To confirm the effect of PlGF on osteoclast formation, bone resorption ex vivo in the presence or absence of PlGF signal transduction=thus without and with antibodies against VEGFR-1 was assessed. Measurement of ⁴⁵Ca-release from cultured tibias was performed as previously described (Engsig et al; 2000 J Cell Biol 151, 87, 879-889). Briefly, on day 1, pregnant females (16 days post coitum) were subcutaneously injected with 100 μCi ⁴⁵Ca. Twenty-four hours later, tibias were isolated and cultured in media supplemented with ascorbate, glutamate and albumin. Right tibias were treated with MF-1 (250 μg/ml/48 hours), left tibias served as controls. Media was changed every day and the amount of radioactivity released in the culture supernatant and remaining in the bones on day 4 of culture was determined. It was shown that Ca-release in organ cultures of embryonic long bones was significantly decreased in PlGF deficient explants. The addition of anti-VEGFR-1 antibodies (250 μg/ml MF-1 reduced osteoclast-mediated ⁴⁵Ca-release by 40% in vitro (% of total radioactivity in supernatant: 7.3±0.5 after IgG versus 4.4±0.3 after MF-1; N=8; P=0.0005). Thus it is shown that the inhibition of PlGF signal transduction by the administration of antibodies against Flt-1 clearly reduces osteoclastic bone resorption.

5. Osteoblast Formation and Differentiation

Although the data show that PlGF affects osteoclast formation, an effect of PlGF on osteoblast formation and differentiation is not excluded. Therefore we studied osteogenic cell growth, differentiation and mineralization in cultures of mesenchymal stem cells derived from PlGF-deficient and WT mice. Cytochemical staining of mesenchymal cell cultures for total colonies, ALP and matrix mineralization showed that osteogenic cell growth and differentiation proceeded similarly in PlGF-deficient and WT mice, indicating that PlGF deficiency does not affect osteoblast function. The decreases in bone formation parameters observed in vivo most likely reflect the low bone turnover in PlGF deficient mice.

1. A pharmaceutical composition comprising:
antibodies against VEGFR-1, wherein said antibodies are capable of inhibiting the signal transduction of VEGFR-1, for the manufacture of a medicament to treat disorders of bone resorption.

2. The pharmaceutical composition of claim 1 wherein said antibody is the monoclonal antibody MF-1 or a humanized version thereof.

3. A method of treating disorders of bone resorption in a subject, said method comprising:
administering to the subject antibodies against VEGFR-1, wherein said antibodies are capable of inhibiting the signal transduction of VEGFR-1.

4. The method according to claim 3 wherein said treatment of disorders of bone resorption is a suppression of bone resorption.

5. The method according to claim 4 wherein the bone resorption is osteoporosis.

6. The method according to claim 3 wherein said antibody is the monoclonal antibody MF-1 or a humanized version thereof.
